(19) United States
(12) Patent Application Publication
(10) Pub. No.: US 2019/0046107 A1
(43) Pub. Date: Feb. 14, 2019

(54) EXERCISE APPLICATION BASED ON MUSCLE STRESS MEASUREMENT

(71) Applicant: Mad Apparel, Inc., Redwood City, CA (US)

(72) Inventors: Seunghee S. Jang, San Francisco, CA (US); Yann J. J. Renard, San Carlos, CA (US); Christopher John Wiebe, Burlingame, CA (US); Dhananja Pradhan Jayalath, San Francisco, CA (US)

(21) Appl. No.: 15/676,918

(22) Filed: Aug. 14, 2017

(51) Int. Cl. A61B 5/00; A41B 1/08; A41D 1/00; A41D 1/08; A63B 24/00; A63B 71/06; A63B 69/00; A61B 5/024

(52) U.S. Cl. CPC A61B 5/486; A61B 2503/10; A41D 1/002; A41D 1/08; A63B 24/0062; A63B 71/06; A63B 69/00; A61B 5/4519; A61B 5/681; A61B 5/024; A61B 5/7278; A41D 2600/10; A63B 2230/60; A63B 2230/06; A63B 2220/40; A63B 2220/803; A63B 2220/75; A63B 2220/836; A41B 1/08

(57) ABSTRACT

An exercise feedback system determines muscle stress measurements using physiological data generated by a sensor-equipped athletic garment. A muscle stress measurement represents an accumulated normalized signal from one or more of the sensors corresponding to a given muscle over a period of time. The exercise feedback system may customize exercise programs, determine risks of injury, or generate biofeedback for presentation on graphical user interfaces using the muscle stress measurements. In an embodiment, the exercise feedback system accesses pre-determined muscle stress measurement models that define criteria for the aforementioned features. For instance, responsive to determining that an athlete is becoming fatigued and exercising with improper form based on a muscle stress measurement, the exercise feedback system modifies the athlete's exercise program to help target and improve the athlete's weaknesses.

### CROSS REFERENCE TO RELATED APPLICATION

This application is related to co-pending U.S. application Ser. No. ______ (Atty. Docket No: 32971-37329/US) entitled “MUSCLE STRESS MEASUREMENT IN A SENSOR EQUIPPED GARMENT,” filed on an even date herewith.

### BACKGROUND

This description generally relates to sensor-equipped athletic garments, and specifically to determining muscle stress measurements using physiological data from the sensors.

Sensors record a variety of information about the human body. For example, electrocardiograph (ECG) electrodes can measure electrical signals from the skin of a person that are used to determine the person's heart rate. In addition, electromyography (EMG) electrodes can measure electrical activity generated by a person's muscles. Heart rate and muscle activation information may be useful for evaluating the person's physiological condition, for instance, while exercising. It is difficult to provide comparable metrics between different muscle groups for a given athlete and between different athletes since the muscle response depends on muscle physiology and body composition, which may vary between muscles and between athletes. A system that generates inconsistent metrics of athletic performance provides a poor user experience, and the athletes may not be able to improve their athletic training with unreliable feedback on exercises.

### SUMMARY

An exercise feedback system determines muscle stress measurements using physiological data generated by a sensor-equipped athletic garment. A muscle stress measurement represents an accumulated normalized signal from one or more of the sensors corresponding to a given muscle or set of muscles over a period of time. By normalizing the physiological data, the exercise feedback system may determine muscle stress measurements, biofeedback, or other metrics for comparison between different muscle groups of an athlete or different athletes. In some embodiments, the exercise feedback system improves the accuracy of muscle stress measurements by classifying the physiological data, for example, based on active or inactive periods of time during an exercise set or program, or based on a type of the exercise.

The exercise feedback system may customize exercise programs, determine risks of injury, or generate biofeedback for presentation on graphical user interfaces using the muscle stress measurements. In an embodiment, the exercise feedback system accesses pre-determined muscle stress measurement models that define criteria for the aforementioned features. For instance, responsive to determining that an athlete is becoming fatigued and exercising with improper form based on a muscle stress measurement, the exercise feedback system modifies the athlete's exercise program to help target and improve the athlete's weaknesses. Additionally, responsive to determining that the athlete's improper form leaves the athlete susceptible to injury, the exercise feedback system notifies the athlete of the risk of injury. In use cases involving a team of athletes, the exercise feedback system may provide biofeedback to a coach of the team that aggregates muscle stress measurement from multiple athletes on the team and highlights athletes that the coach should be aware of due to inefficiencies or potentially injury risk.

### BRIEF DESCRIPTION OF DRAWINGS

FIG. 1 is a diagram of a system environment for determining muscle stress measurements according to one embodiment.

FIG. 2 is a diagram of a sensor-equipped athletic garment according to one embodiment.

FIG. 3 is a block diagram of an exercise feedback system according to one embodiment.

FIG. 4 is a diagram of a user interface showing biofeedback for different muscles according to one embodiment.

FIG. 5 is a diagram of a user interface showing biofeedback for left and right muscles of the body according to one embodiment.

FIG. 6 is a diagram of a user interface showing biofeedback for a team of athletes according to one embodiment.

FIG. 7 is a flowchart of a process for determining muscle stress measurements according to one embodiment.

FIG. 8 is a flowchart of a process for using a muscle stress measurement model according to one embodiment.

The figures depict embodiments of the present invention for purposes of illustration only. One skilled in the art will readily recognize from the following discussion that alternative embodiments of the structures and methods illustrated herein may be employed without departing from the principles of the invention described herein.

### DETAILED DESCRIPTION

### I. System Overview

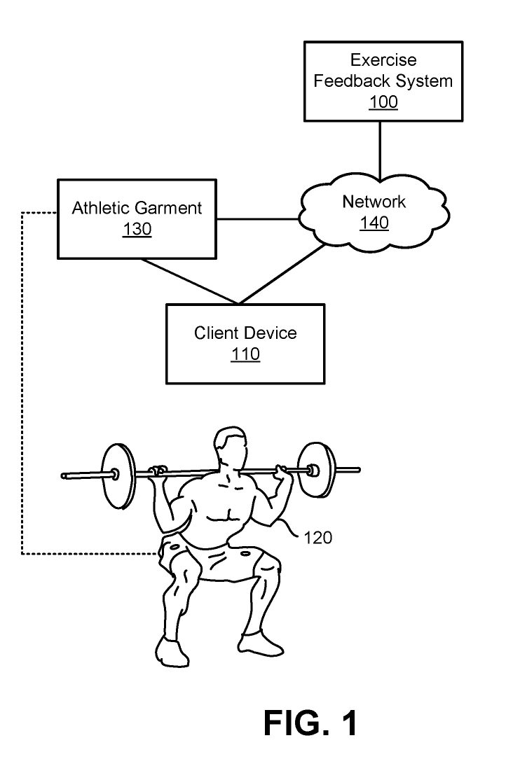

FIG. 1 is a diagram of a system environment for monitoring exercise data according to one embodiment. The system environment includes an exercise feedback system 100, client device 110, and athletic garment 130 communicatively coupled together via a network 140. Users 120 of the exercise feedback system 100 are also referred to herein as “athletes.” In other embodiments, different and/or additional entities can be included in the system architecture.

The client device 110 is a computing device capable of receiving user input as well as transmitting and/or receiving data via the network 140. A client device is a device having computer functionality, such as a smartphone, personal digital assistant (PDA), a mobile telephone, tablet, laptop computer, desktop computer, a wearable computer (such as a smart watch, wrist band, arm band, chest band, or the like), or another suitable device. In one embodiment, a client device executes an application allowing a user of the client device to interact with the exercise feedback system 100. For example, a client device executes a browser application to enable interaction between the client device and the exercise feedback system 100 via the network 140. In another embodiment, a client device 110 interacts with the exercise feedback system 100 through an application programming interface (API) running on a native operating system of the client device, such as IOS® or ANDROID™.

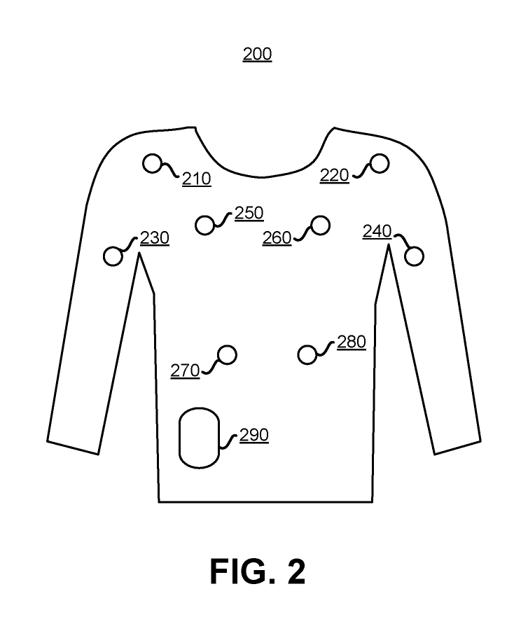

An athlete 120 wears the athletic garment 130 (further described below with reference to FIG. 2) while performing exercises. The athletic garment 130 records physiological data, e.g., muscle activation data or heart rate data, of the athlete. Based on the physiological data, the exercise feedback system 100 generates muscle stress measurements or biofeedback customized for the athlete. The muscle stress measurements may correspond to any muscle, portion of a muscle, or set of muscles of the athlete. For example, the athlete's biceps may include a left and right biceps muscle, and the biceps may be part of a group of upper body muscles. As described herein, a muscle includes soft tissue for the purpose of producing force or motion of the human body, and may be a slow twitch or fast twitch type of muscle. Further, a coach of the athlete can view the biofeedback on a client device 110 and provide additional feedback for the athlete. In some embodiments, the exercise feedback system 100 can communicate feedback using audio output or haptic feedback of the client device 110.

### II. Example Athletic Garment

FIG. 2 is a diagram of a sensor-equipped athletic garment 200 according to one embodiment. The athletic garment 200 includes sensors that contact the skin of an athlete wearing the athletic garment 200. For example, the sensors can be electrodes that measure electromyography (EMG) signals (electrical signals caused by muscle cells) also referred to as muscle activation data or muscle activation levels, electrocardiograph (ECG) signals (electrical signals caused by depolarization of the user's heart muscle in particular) also referred to as heart rate data, electrical signals modified by the tissue of the user (also referred to as bioimpedance data), electroencephalograph (EEG) signals, magnetoencephalograph (MEG) signals, among other types of signals associated with physiological data. The sensors may also include other types of sensors such as accelerometers and gyroscopes (which generate motion data based on the athlete's movement), temperature sensors, pressure sensors, humidity sensors, geographical location sensors, etc. The sensors generate physiological data of the athlete based on the measured signals. The sensors are communicatively coupled to a processing unit 290. The processing unit 290 can aggregate and analyze the physiological data from the sensors. The processing unit 290 can also provide the physiological data to the client device 110 or exercise feedback system 100 via the network 140.

In the embodiment shown in FIG. 2, the athletic garment 200 includes eight sensors that record muscle activation data from the athlete's muscles nearby each sensor. In particular, sensors 210 and 220 located on the right and left shoulder of the athletic garment 200 can record muscle activation data of the athlete's deltoid muscles. Sensors 230 and 240 located on the right and left sleeves of the athletic garment 200 can record muscle activation data of the athlete's triceps and/or bicep muscles. Sensors 250 and 260 located on the right and left chest of the athletic garment 200 can record muscle activation data of the athlete's pectoral muscles. Sensors 270 and 280 located on the right and left abdomen of the athletic garment 200 can record muscle activation data of the athlete's abdominal and oblique muscles. Though the athletic garment 200 shown in FIG. 2 includes eight sensors and the processing unit 290, in other embodiments, the athletic garment 200 can include any number of sensors or other types of components or electronics at any location or configuration within the athletic garment 200.

It should be noted that while the athletic garment 200 shown in FIG. 2 is a long sleeve shirt, the principles described herein apply equally to any garment, including but not limited to a short sleeved shirt, a tank top, pants, shorts (e.g., the athletic garment 130 shown in FIG. 1), or any other suitable garment. In embodiments where the athletic garment is a pant or shorts, sensors of the athletic garment can record muscle activation data from muscles on an athlete's lower body, e.g., inner and outer quadriceps (also referred to as “quads”), gluteus maximus (also referred to as “glutes”), hamstrings, calves, and the like.

### III. Example Exercise Feedback System

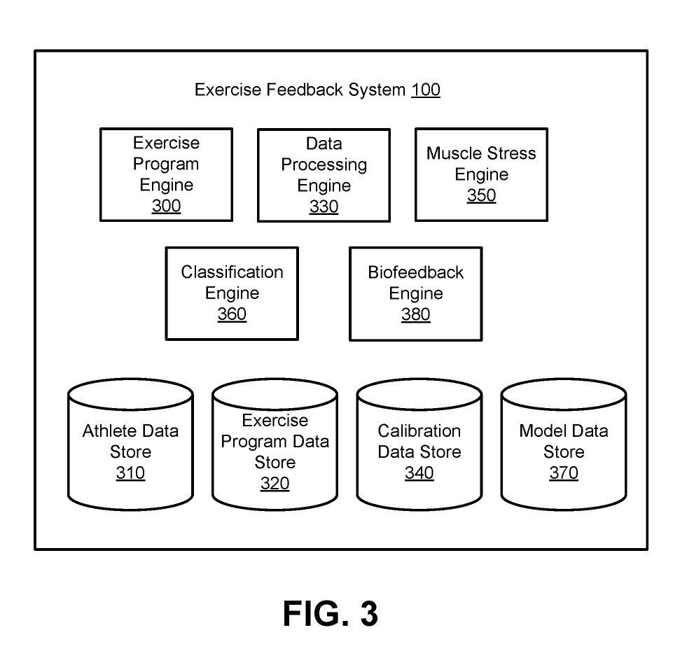

FIG. 3 is a block diagram of the exercise feedback system 100 according to one embodiment. The exercise feedback system 100 includes an exercise program engine 300, athlete data store 310, exercise program data store 320, data processing engine 330, calibration data store 340, muscle stress engine 350, classification engine 360, model data store 370, and biofeedback engine 380. In other embodiments, the exercise feedback system 100 may include additional, fewer, or different components for various applications, which are not shown for purposes of clarity.

The exercise program engine 300 manages exercise programs for athletes of the exercise feedback system 100. In some embodiments, an athlete joins the exercise feedback system 100 by completing an onboarding process during which the exercise program engine 300 receives user information and physiological data associated with the athlete and stores the user information and physiological data in the athlete data store 310. User information includes, for example, demographic data (e.g., age, gender, ethnicity, etc.), geographic data, exercise related data (e.g., sports played, a specific position for sport, or sports team information), or physiological data (e.g., height or weight). Physiological data may include data generated by the sensors of athletic garment 200 while the user performs prescribed onboarding baseline exercises. The sensor data generated during the onboarding baseline may be used to set calibration parameters or reference levels for each muscle group such that subsequent metrics for that muscle can be reported as a relative percentage from the onboarding baseline. Calibration parameters are stored in the calibration data store 340. Exercise programs may include various exercises or other athletic related activities (e.g., diet or medical health data), and the exercise program engine 300 may customize the types and number of exercises for a given athlete based on user information retrieved from the athlete data store 310.

The exercise program engine 300 may generate or update exercise programs based on information from the exercise program data store 320, which includes a database of exercises and other types of athletic training information or guidelines. An exercise program may include exercise sets (also referred to as a “set”), where a set includes a number of repetitions of an exercise to be performed in sequence. As an example use case, the exercise program engine 300 determines that a given athlete is a basketball player aiming to increase their vertical jump height. Thus, the exercise program engine 300 updates an exercise program for the given athlete that includes exercises for developing lower body strength such as squats and calf raises. Moreover, the exercise program engine 300 may determine a suitable weight for the exercises and update the weight over time based on the athlete's performance, e.g., responsive to determining that the athlete has increased strength, the exercise program engine 300 increases the amount of weight to lift per repetition of an exercise. By personalizing exercise programs, the exercise program engine 300 determines strengths and weaknesses specific to an athlete, which helps improve the effectiveness of the athlete's training.

The data processing engine 330 processes physiological data generated by sensors of an athletic garment (e.g., athletic garment 200 shown in FIG. 2) for determining muscle stress measurements. The data processing engine 330 can receive the physiological data from the client device 110 or the processing unit 290 of the athletic garment 200. In some embodiments, the data processing engine 330 pre-processes the physiological data by performing noise filtering techniques. The physiological data may vary based on parameters associated with the athlete wearing the athletic garment, e.g., differences in the physiology between multiple types of muscles influences the physiological data representing contraction of the muscles. For instance, the glutes and quads differ in the number of muscle fibers, fiber size, fiber type distribution (e.g., slow twitch or fast twitch), and thickness of adipose tissue between the muscle tissue and skin surface. The amplitude of physiological data (e.g., muscle activation level) may be proportional to the muscle fiber size and/or inversely proportional to the amount of fat between the muscle and the portion of skin.

Additionally, physiological data generated by the sensors may vary due to athlete-to-athlete differences in physiological traits, sensor contact quality, among other factors. For instance, thicker hair on the athlete's skin or skin dryness may result in poor sensor contact quality, and thus reduce the amplitude of physiological data generated by the sensors and increase the amplitude of measured noise. As another example, skin oiliness or sweat may improve sensor contact quality, and thus increase the amplitude of physiological data generated by the sensors and reduce the amplitude of measured noise. The data processing engine 330 may determine a quality of physical contact between a sensor and the athlete's skin based on bioimpedance data, which is generated by another sensor or the same sensor. In some embodiments, the amplitude of physiological data generated by a sensor and the signal to noise ratio is inversely proportional to the bioimpedance between the sensor and the skin of the athlete.

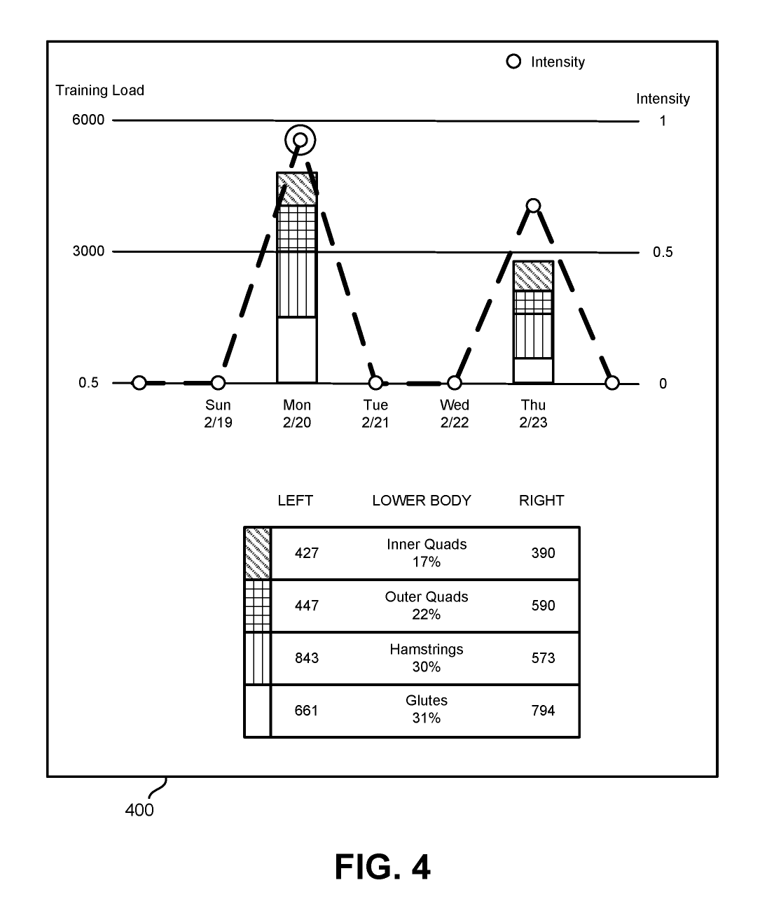

The data processing engine 330 normalizes physiological data between different types of muscles and different athletes to account for the above described sources of physiological data variation. Using normalized physiological data, the exercise feedback system 100 may provide biofeedback for a given type of muscle relative to other types of muscles. For example, as described below with reference to FIG. 4, the exercise feedback system 100 may provide a graphical user interface including a comparison of muscle stress (labeled as “training load” in FIG. 4) for multiple lower body muscles. Further, the data processing engine 330 may perform normalization using calibration parameters retrieved from the calibration data store 340. As an example, the glutes and quads have different calibration parameters, which are used by the data processing engine 330 to adjust raw amplitude values of the physiological data received for the two types of muscles (e.g., for comparison on the same axis as shown in FIG. 4). The data processing engine 330 may normalize physiological data by scaling the physiological data for a muscle corresponding to one of the sensors of an athletic garment 200 by a calibration parameter. In some embodiments, the data processing engine 330 generates a pre-processed EMG signal of the physiological data before normalization. For instance, the data processing engine 330 determines a power representation of the EMG signal by using averaging, a linear envelope, rolling root mean square or any other suitable technique to generate a pre-processed EMG signal. Additional filtering may be applied to the pre-processed signal to further filter and reduce potential noise sources. In an embodiment, the amplitude of the pre-processed EMG signal represents the power of contraction at a point in time, and the data processing engine 330 can determine the relative level of contraction by normalizing the pre-processed signal using the calibration parameters.

The muscle stress engine 350 determines a muscle stress measurement for a muscle by accumulating the normalized physiological data from the data processing engine 330. In an embodiment, the muscle stress engine 350 determines a start and stop time bound over which the muscle stress measurement is to be calculated, and the muscle stress engine 350 accumulates the normalized physiological data over the time bound. The start and stop time bound may indicate the start and end of a set or repetition. For example, the exercise feedback system 100 instructs an athlete to perform a set of squats including ten repetitions during an exercise program. By determining the time bound, the muscle stress engine 350 avoids accumulating physiological data generated by the sensors when the athlete is not performing the exercises, e.g., while resting between sets of exercises. Thus, the muscle stress engine 350 may determine a more accurate measurement of the muscle stress for the set by not accumulating physiological data generated outside of the time bound.

In some embodiments, the muscle stress engine 350 accumulates normalized physiological data based on classifications determined by the classification engine 360. As an example, the classification engine 360 classifies normalized physiological data by determining a period of time during which an athlete actively performed an exercise or periods of time during which the athlete did not actively perform the exercise. The classification engine 360 can determine these periods of time without requiring manual input from the athlete. For instance, the classification engine 360 uses physiological data generated by the EMG sensors and motion data received from motion sensors of the athletic garment to determine whether the athlete is actively training, performing exercises, or performing a particular type of exercise. Based on the type of classification, the muscle stress engine 350 may perform active segmentation of sensor data received from the athletic garment, e.g., to provide exercise metrics and biofeedback. Similar to the time bound feature described above, the muscle stress engine 350 may improve the accuracy of muscle stress measurements by accumulating normalized physiological data received (or generated) during the active periods of time and not the inactive periods of time. In addition to resting between sets, an athlete may also briefly rest between repetitions or otherwise temporarily interrupt the exercise, e.g., to adjust the athletic garment. Classifying the active and inactive periods of time helps the muscle stress engine 350 account for these discrepancies in the physiological data.

In some embodiments, the classification engine 360 determines active or inactive periods of time for a given muscle or type of exercise. As an example, while an athlete is performing squat exercises, the classification engine 360 classifies the received sensor data to differentiate between physiological data for upper body muscles and lower body muscles. Since squat exercises are intended to train the lower body muscles, the classification engine 360 may determine active periods of time when the received physiological data for the lower body muscles has an amplitude or accumulated muscle stress greater than a threshold value. On the other hand, the classification engine 360 may process received physiological data for the upper body muscles when the athlete is performing exercises such as pullups, overhead lifts, pushups, or curls. In some embodiments, the classification engine 360 disregards physiological data—for determining muscle stress measurements of a particular muscle—generated during a given exercise responsive to determining that the given exercise is not directed to training that particular muscle. For instance, physiological data of the glutes and quads generated while the athlete is performing certain upper body exercises may not be normalized or accumulated for determining muscle stress measurements of the lower body muscles.

The classification engine 360 may also classify normalized physiological data based on quality of physical contact between a sensor and the athlete's skin. In particular, responsive to determining that the quality of physical contact during a period of time is less than a threshold quality (e.g., indicative of poor contact quality), the muscle stress engine 350 may exclude or modify the physiological data received during the period of time from accumulation for determining muscle stress measurement. Other types of classification include, e.g., determining whether the received physiological data corresponds to a low, medium, or high intensity level of exercise; aerobic or anaerobic exercise; or a power, strength, endurance, speed, or hypertrophy type of exercise.

In some embodiments, the muscle stress engine 350 modifies or interprets physiological data received from a sensor over periods of poor contact quality, as classified by the classification engine 360. For example, if the contact quality is intermittently poor over short periods, the muscle stress engine 350 interprets physiological data during these short periods using sensor data received during other periods of good contact quality as boundary conditions for linear or polynomial curve fitting. Additionally, physiological data corresponding to sensors with poor contact quality (e.g., less than a given threshold) may be modified or interpreted by the muscle stress engine 350 using data from other sensors with good contact quality (e.g., equal to or greater than the given threshold). For example, when determining a stress measurement for the right hamstring muscle during a period of poor contact quality, the muscle stress engine 350 may use alternate physiological data corresponding to the left hamstring muscle, responsive to determining that the alternate physiological data has good contact quality. Additionally, the muscle stress engine 350 may combine physiological data from multiple sensors to determine an approximate data set during a period of poor contact quality.

The muscle stress engine 350 may determine muscle stress measurements for a group of muscles. For instance, the muscle stress engine 350 accumulates the stress for both a group of lower body muscles and a group of upper body muscles. Additionally, the muscle stress engine 350 may accumulate stress for groups of muscles of the left and right sides (or anterior and posterior sides) of symmetry of the athlete's body. In some embodiments, the muscle stress engine 350 determines muscle stress measurements based on historical muscle stress measurements of exercises previously performed by the athlete. The historical muscle stress measurements may indicate a pattern of performance such as an increase or decrease over time of the strength of a given muscle of the athlete. Further, the muscle stress engine 350 may determine a fatigue state of the athlete based on the pattern of performance, magnitude of a muscle stress measurement, or a frequency of a muscle stress measurement.

The exercise program engine 300 modifies an exercise program based on a muscle stress measurement determined by the muscle stress engine 350, in one embodiment. In particular, the muscle stress engine 350 accesses a pre-determined muscle stress measurement model (also referred to herein as a “model”) stored in the model data store 370. The model defines one or more criteria for modifying the exercise program. For example, a criteria may be satisfied by the muscle stress engine 350 determining that a muscle stress measurement of an athlete exceeds a threshold deviation relative to athlete population norms, e.g., based on aggregate data retrieved from the athlete data store 310. Specifically, the model may indicate a target motion profile (e.g., indicative of proper form of an exercise to avoid injury) or muscle stress profile (e.g., indicating a change in muscle exertion as an athlete lifts a weight), which may be customized based on the athlete's demographic or physiological data. If the athlete exceeds the threshold deviation, the muscle stress engine 350 may determine that the exercise program is currently too challenging (e.g., resulting in improper exercise form) or not challenging enough (e.g., resulting in under-utilization of muscles) for the athlete. Thus, responsive to the muscle stress engine 350 determining that the criteria is satisfied by the muscle stress measurement, the exercise program engine 300 may adjust the difficulty of the exercise program to provide an appropriate level of challenge to the athlete.

The exercise program engine 300 may also modify an exercise program based on an imbalance of muscle stress associated with a criteria of a pre-determined muscle stress measurement model. The muscle stress engine 350 may determine the imbalance by determining that the muscle stress of the left body side or right body side is greater than the muscle stress of the other side by at least a threshold difference. Responsive to the muscle stress engine 350 determining that the criteria is satisfied by an athlete's muscle stress measurement, the exercise program engine 300 may add or remove an exercise from the exercise program to address the imbalance. For instance, the exercise program engine 300 adds exercises to increase the muscle stress of the “weaker” side of the body that currently has lower muscle stress.

Further, the exercise program engine 300 may modify exercise programs based on other factors such as a fatigue level of an athlete, which may be determined based on historical muscle stress measurement. For instance, responsive to determining that the fatigue level is greater than a threshold level (e.g., indicative of chronic fatigue), the exercise program engine 300 removes exercises from the exercise program to alleviate the stress experienced by the athlete and mitigate injury risk from over training. In addition, the exercise program engine 300 may reduce the number or training stress of exercises during a period of time prior to an identified high stress event in the exercise program such as an athletic competition at which the athlete plans to participate. The exercise program engine 300 may provide a notification to an athlete describing a modification to an exercise program as well as relevant context. For example, the exercise program engine 300 notifies the athlete that chronic fatigue is detected and that the number of exercise sessions per week will be reduced accordingly, e.g., to mitigate injury risk. In some embodiments, the exercise program engine 300 presents an exercise program modification in a graphical user interface, e.g., highlighting a updated number of repetitions, sets, or weight for an exercise in a list, timeline, or calendar display of the athlete's exercise program. Additionally, the exercise program engine 300 may dynamically reformat or resize components of a graphical user interface based on modifications. For instance, a calendar display shows graphics (e.g., a window indicating a number of reps and weight for a particular type of exercise) corresponding to exercises for completion by the athlete on various days or times of the week. Responsive to a modification, the exercise program engine 300 removes existing graphics (e.g., with a cross mark or a faded color) or adds new graphics to the calendar (e.g., which may be emphasized in bold, with brighter colors, or with enlarged size).

Since the exercise program engine 300 can dynamically modify an exercise program and provide a notification of the modification while the athlete is performing the exercise program, the exercise feedback system 100 improves the athlete's training in real-time, instead of having the athlete or a coach review the athlete's performance after an exercise workout or session has been completed. Additionally, the exercise feedback system 100 may maintain accurate and continuous accumulation of physiological data even as the exercise program is modified. In particular, responsive to increasing the weight lifted per repetition of a squat exercise, the muscle stress engine 350 determines muscle stress measurements subsequent to the modification based on the increased weight rather than an initial weight per repetition. The exercise feedback system 100 may automatically update physiological data normalization or classifications for any number or type of modification of an exercise program, which allows athletes and coaches to receive reliable metrics and biofeedback while training.

In a “return to play” use case when an athlete is undergoing rehabilitation after an injury or other type of physical treatment, the exercise program engine 300 may increase training stress of the exercise program responsive to determining that an athletic metric of the athlete improved over a duration of time. For example, the muscle stress engine 350 determines that the athlete's lower body strength or mobility improved based on analyzing trends in historical muscle stress measurements of the athlete. The exercise program engine 300 may gradually increase the training stress to reduce the likelihood of aggravating a healing injury. On the other hand, responsive to determining that the athlete's metrics are not improving as expected or do not satisfy a training goal (e.g., lift a certain volume of weights or repetitions), the exercise program engine 300 may modify the exercise program accordingly, or may take additional action such as notify the athlete or a coach that the athlete may have a risk of injury. Additionally, the exercise program engine 300 may provide remedial information to the athlete or coach such as recommended exercise routines and practices to alleviate the risk of injury. Moreover, based on the feedback from the exercise program engine 300, a coach may adjust an athlete's training program, e.g., to set a new target timeline before the athlete is ready to return to play.

In some embodiments, the exercise feedback system 100 uses the models to perform other functionality such as providing biofeedback by the biofeedback engine 380. The biofeedback engine 380 may generate or update various graphical user interfaces (examples of which are shown in FIGS. 4-6) to present biofeedback via a client device 110. As an example, a model defines a criteria for determining a risk of injury. Responsive to the muscle stress engine 350 determining that the criteria is satisfied by an athlete's muscle stress measurement, the biofeedback engine 380 notifies the athlete of the risk of injury. In some embodiments, the biofeedback engine 380 communicates the risk of injury via a push notification presented by a client device 110 while an athlete is exercising, so that the athlete can promptly stop or adjust an exercise to avoid the injury. Further, an athletic garment worn by the athlete may also communicate the notification by providing haptic feedback, e.g., vibrating at a certain position on the athlete's body during an exercise to alert the athlete to reduce the weight being lifted or to correct improper form of an exercise. The biofeedback engine 380 may provide context with a notification of injury risk, for example, indicating a type of risk (e.g., straining a muscle or wearing out a joint), severity of the risk, or remedial action such as performing stretches, icing or heating a muscle, resting, or switching to a different type of exercise. In an example, the risk of injury may be determined based on an identified imbalance of muscle stress or a fatigue level of the athlete, and the biofeedback engine 380 provides context informing the athlete to increase training on the left or right side upper arm muscles (e.g., to address the identified imbalance) or reduce the amount of weights overall to recover from a high-risk fatigue level and avoid injury.

In addition to providing the notification of injury risk, the biofeedback engine 380 may also generates and provide other types of biofeedback for athletes based on the muscle stress measurements. The biofeedback indicates, for example, a metric of athletic performance such as a percentage value, a Boolean value (e.g., satisfactory or unsatisfactory), or any suitable type of value. In some embodiments, the biofeedback indicates a measure of progress of an athlete over a period of time and is generated based on context describing the corresponding exercise (e.g., using information from the athlete data store 310 or exercise program data store 320). The context may include a type of exercise, or a volumetric load based on a product of a number of repetitions and corresponding weights lifted by an athlete. The biofeedback may be generated based on heart rate data captured by sensors of an athletic garment or associated with a particular muscle, muscle group, repetition, or set. Based on normalized physiological data, the biofeedback engine 380 may generate biofeedback including a comparison between contributions of a set of muscles with that of a different set of muscles of the athlete while performing an exercise.

In some embodiments, the biofeedback engine 380 generates a graphical depiction of muscles of an athlete. In particular, the biceps and quads (among other types of muscles) may be overlaid on the arm and leg portions, respectively, of a human body graphic (e.g., resembling a silhouette, avatar, or the like) of the athlete. The biofeedback engine 380 may present muscle stress measurements, biofeedback, or other metrics the muscles by dynamically updating a color or size of the graphic depiction of the corresponding muscle. For instance, as the muscle stress measurement increases, the graphic of the muscle becomes a brighter color or increases in size to illustrate that the muscle is being contracted for an exercise. Thus, the athlete can view a real-time progression of multiple muscles that increase or decrease in stress or activation levels throughout stages of an exercise, e.g., the athlete utilizes the arm and leg muscles for different motions of an Olympic lift.

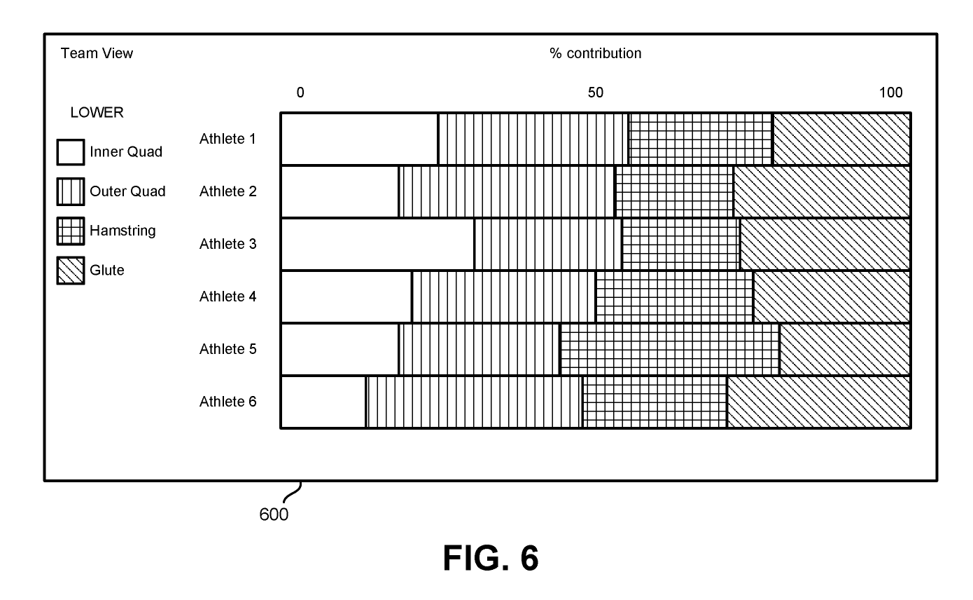

As an example use case, an athlete using the exercise feedback system 100 is part of an athletic team including multiple athletes, coaches, or other personnel. The biofeedback engine 380 may generate biofeedback including a comparison between the athlete and another athlete of the same team (e.g., as shown in FIG. 6). In some embodiments, the biofeedback presents a comparison of athletes for a particular demographic range or athletic skill level (e.g., amateur, college, or professional). Furthermore, the biofeedback engine 380 may provide the biofeedback for presentation to a coach of the team, and may customize graphical user interfaces to display the biofeedback, e.g., the coach is presented with additional biofeedback aggregating performance of the team that is not necessarily presented to individual athletes on the team.

In some embodiments, some or all of the functionality of the exercise feedback system 100 may be performed by or implemented within a client device 110. For example, the client device 110 stores pre-determined muscle stress measurement models and uses a local muscle stress engine to determine whether a model's criteria for modifying an exercise program is satisfied. This can be advantageous because the client device 110 may not always have a network connection while an athlete is exercising (e.g., the athlete's gym does not have internet available). Thus, muscle stress measurements are determined and analyzed locally on the client device 110 without having to upload the physiological data to the exercise feedback system 100 for processing.

### IV. Example User Interfaces

FIG. 4 is a diagram of a user interface 400 showing biofeedback for different muscles according to one embodiment. The biofeedback engine 380 generates the user interface 400 including a graph and numerical metrics displaying muscle stress measurements of an athlete's lower body muscles such as inner quads, outer quads, hamstrings, and glutes, over several days. Based on the physiological data normalized by the data processing engine 330, the muscle stress engine 350 may determine the training load and intensity (e.g., derived from muscle stress measurements) of each of the muscles for presentation on a same axis (or axes). In an embodiment, the training load represents a metric of muscle stress, where one unit of training load may correspond to the stress accumulated by one second of contraction of a muscle, e.g., at a level of a baseline calibration parameter. Further, the intensity may be determined based on training load per second, which indicates the energy expended or stress on a muscle as a measure of power of contraction. For example, metrics for power-targeted exercises (e.g., lifts) may have a greater intensity and lower training load relative to corresponding metrics of hypertrophy-targeted exercises. Thus, the athlete may view performance of the muscles relative to each other, which may assist the athlete or a coach of the athlete in evaluating and improving the athlete's exercise program or other training. For example, using the biofeedback presented on the user interface 400, the athlete may identify that the training loads on Monday of the hamstrings and glutes are greater relative to those of the inner and outer quads. Accordingly, the athlete may adjust the exercises on Thursday (e.g., based on a modification of the athlete's exercise program by the exercise program engine 300) to improve the balance of training stress among the various muscles.

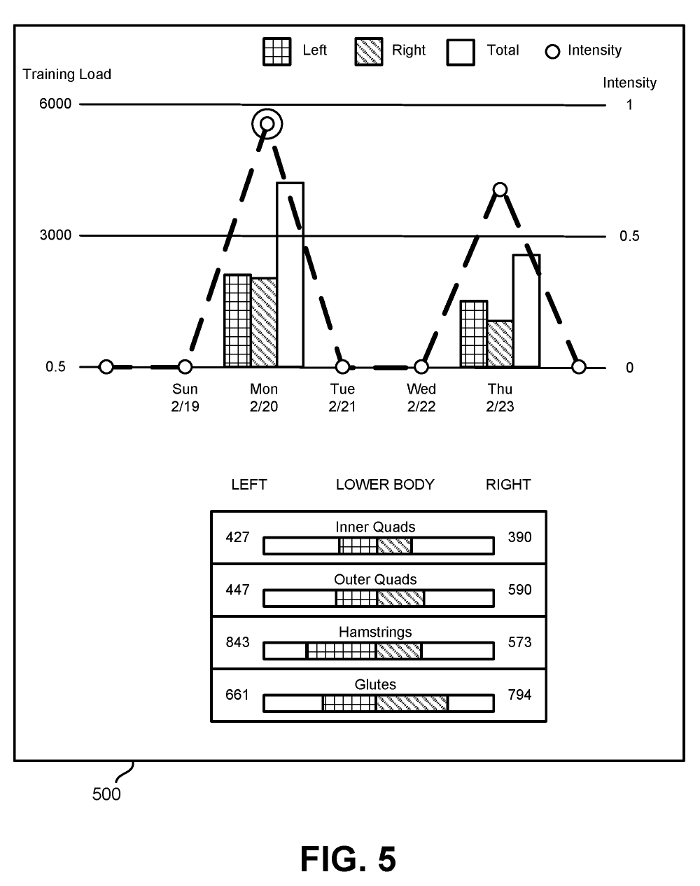

FIG. 5 is a diagram of a user interface 500 showing biofeedback for left and right muscles of the body according to one embodiment. Similar to the user interface 400 shown in FIG. 4, the user interface 500 includes a graph and numerical metrics displaying muscle stress measurements of an athlete's lower body muscles. Moreover, the user interface 500 presents a comparison of the training load between the left and right lower body muscles of the athlete. In particular, based on the presented biofeedback, the athlete may identify an imbalance between the training loads on one side of the body relative to the other. The imbalance may be presented as a difference in heights in the bar graph or numerical values as shown in FIG. 5. In other embodiments, the imbalance may be displayed as a difference in colors, percentages, or other graphical elements, for example, by updating colors of various muscles in a depiction of a human body to illustrate a greater level of muscle stress in one set of muscles relative to another.

FIG. 6 is a diagram of a user interface 600 showing biofeedback for a team of athletes according to one embodiment. The biofeedback engine 380 generates the user interface 600 by aggregating muscle stress measurements from multiple athletes of the team for athlete-to-athlete comparisons. In particular, the “Team View” graph shown in FIG. 6 displays the athletes' percentages of muscle contribution of the various lower body muscles on the same axis. Thus, a coach of the team can use the biofeedback to evaluate the overall performance of the team as well as analyze the performance of a given athlete relative to another. For example, the biofeedback may show trends such as a first athlete increasing muscle contribution of the inner and outer quads, while a second athlete increases muscle contribution of the hamstrings and glutes, e.g., due to differences in the exercise programs of the two athletes. The biofeedback engine 380 may determine statistics of athletic performance to identify athletes who are falling behind in training, relative to other athletes on the same team. In some embodiments, the biofeedback engine 380 determines an average of the muscle stress measurements of multiple athletes on a team accumulated over a period of time (e.g., a week or month). Responsive to determining that a given athlete on the team has a muscle stress measurements that deviates from the average by at least a threshold value (e.g., one or more standard deviations), the biofeedback engine 380 may notify a coach of the team of this outlier and provide recommendations for remedying the given athlete's training to more closely match the average performance of the team. For example, the biofeedback indicates that the given athlete should participate in additional 1-on-1 strength training exercise workouts, and the exercise program engine 300 may modify the given athlete's exercise program to according to the biofeedback.

### V. Example Process Flows

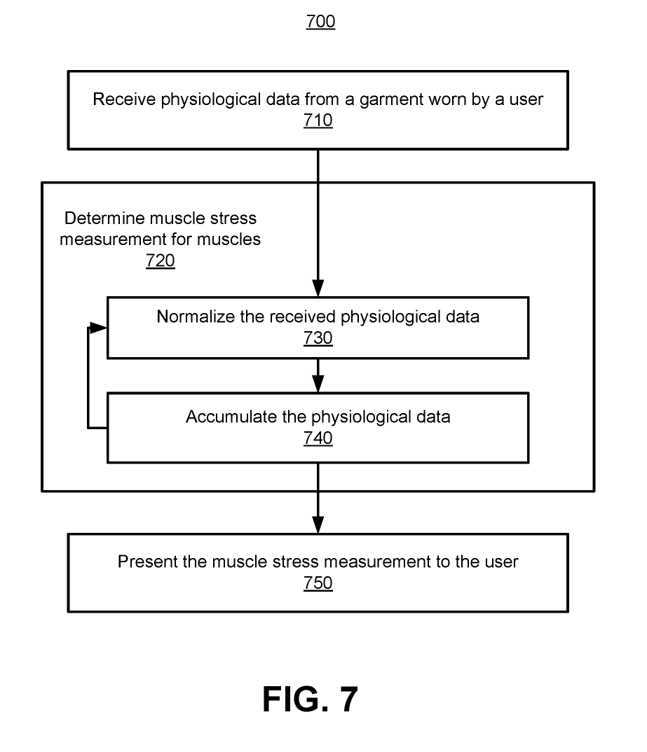

FIG. 7 is a flowchart of a process 700 for determining muscle stress measurement according to one embodiment. In some embodiments, the process 700 is performed by the exercise feedback system 100—e.g., modules of the exercise feedback system 100 described with reference to FIG. 3—within the system environment in FIG. 1. The process 700 may include different or additional steps than those described in conjunction with FIG. 7 in some embodiments or perform steps in different orders than the order described in conjunction with FIG. 7.

In one embodiment, the data processing engine 330 receives 710 physiological data from an athletic garment worn by a user (athlete), where the physiological data describes muscle activation of muscles of the user while performing an exercise. The muscle stress engine 350 determines 720, for each of the muscles, a muscle stress measurement for the exercise based on the received physiological data. Specifically, the data processing engine 330 normalizes 730 the physiological data corresponding a given muscle relative to the physiological data corresponding to one or more other muscles. Additionally, the muscle stress engine 350 accumulates 740 the normalized physiological data. The exercise feedback system presents 750 the muscle stress measurement to the user, e.g., by displaying biofeedback generated by the biofeedback engine 380 via a client device 110.

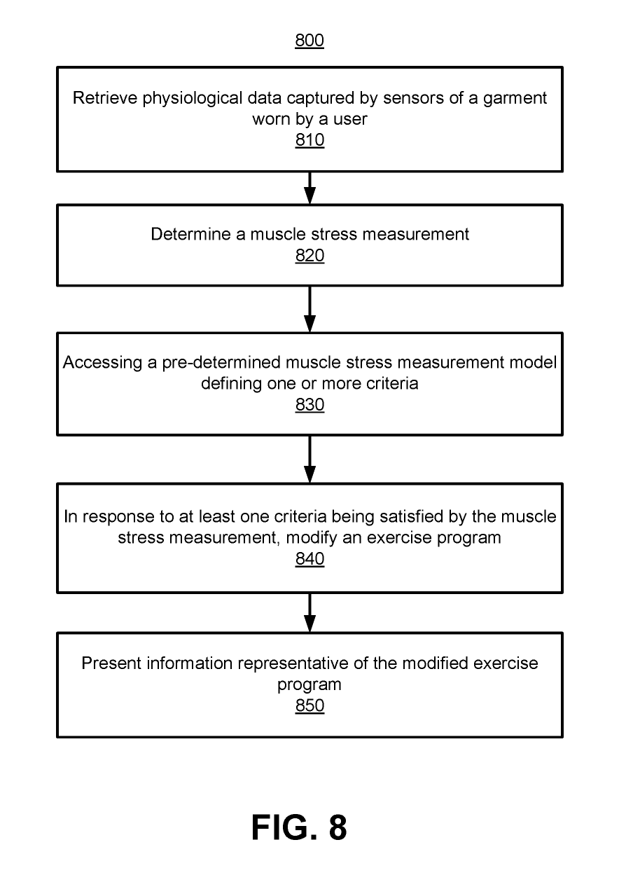

FIG. 8 is a flowchart of a process 800 for using a muscle stress measurement model according to one embodiment. In some embodiments, the process 800 is performed by the exercise feedback system 100—e.g., modules of the exercise feedback system 100 described with reference to FIG. 3—within the system environment in FIG. 1. The process 800 may include different or additional steps than those described in conjunction with FIG. 8 in some embodiments or perform steps in different orders than the order described in conjunction with FIG. 8.

In one embodiment, the data processing engine 330 retrieves 810 physiological data captured by sensors of an athletic garment worn by a user (athlete) while performing an exercise using a set of muscles for an exercise program presented to the user by a client device 110. The muscle stress engine 350 determines 820 a muscle stress measurement for the set of muscles that is representative of a magnitude of exertion by the set of muscles during the exercise (e.g., using the process 700 shown in FIG. 7). The muscle stress engine 350 accesses 830 a pre-determined muscle stress measurement model defining one or more criteria for modifying the exercise program. In response to at least one of the criteria being satisfied by the muscle stress measurement, the exercise program engine 300 modifies 840 the exercise program. The exercise program engine 300 presents 850 information representative of the modified exercise program by the client device 110. In other embodiments, the pre-determined muscle stress measurement model defines criteria for determining risk of injury of the user. Responsive to the criteria being satisfied, the biofeedback engine 380 may provide a notification of the risk of injury to the user.

### VI. Additional Considerations

The foregoing description of the embodiments of the invention has been presented for the purpose of illustration; it is not intended to be exhaustive or to limit the invention to the precise forms disclosed. Persons skilled in the relevant art can appreciate that many modifications and variations are possible in light of the above disclosure.

Some portions of this description describe the embodiments of the invention in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In one embodiment, a software module is implemented with a computer program product including a computer-readable non-transitory medium containing computer program code, which can be executed by a computer processor for performing any or all of the steps, operations, or processes described.

Embodiments of the invention may also relate to a product that is produced by a computing process described herein. Such a product may include information resulting from a computing process, where the information is stored on a non-transitory, tangible computer readable storage medium and may include any embodiment of a computer program product or other data combination described herein.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention, which is set forth in the following claims.

What is claimed is:

1. A method comprising:
receiving physiological data captured by sensors of a garment worn by a user, the physiological data describing muscle activation of a set of muscles of the user while performing an exercise in an exercise program presented to the user by a client device;
determining, for the set of muscles, a muscle stress measurement representative of a magnitude of exertion by the set of muscles during the exercise;
accessing a pre-determined muscle stress measurement model, the pre-determined muscle stress measurement model defining one or more criteria for modifying the exercise program;
in response to at least one criteria defined by the pre-determined muscle stress measurement model being satisfied by the muscle stress measurement, modifying the exercise program; and
presenting to the user, by the client device, information representative of the modified exercise program.

2. The method of claim 1, further comprising:
determining that the at least one criteria defined by the pre-determined muscle stress measurement model is satisfied by determining that muscle stress measurement exceeds a threshold deviation relative to athlete population norms.

3. The method of claim 1, further comprising:
determining an imbalance of muscle stress of the user in response to the at least one criteria defined by the pre-determined muscle stress measurement model being satisfied by the muscle stress measurement; and
wherein modifying the exercise program comprises adding or removing one or more exercises of the exercise program to address the imbalance of muscle stress.

4. The method of claim 3, wherein determining the imbalance of muscle stress comprises:
determining that muscle stress of a left body side or right body side is greater than muscle stress of the other side by at least a threshold difference; and
wherein modifying the exercise program comprises adding the one or more exercises to increase the muscle stress of the other side.

5. The method of claim 1, wherein modifying the exercise program comprises:
removing one or more exercises of the exercise program in response to determining that a fatigue level of the user is greater than a threshold level, the fatigue level determined based on historical muscle stress measurement of the user.

6. The method of claim 1, wherein modifying the exercise program comprises:
identifying a high stress event in the exercise program; and
reducing training stress of the exercise program to during a period of time prior to the high stress event.

7. The method of claim 1, wherein modifying the exercise program comprises:
determining an athletic metric based on historical muscle stress measurement of the user; and
increasing training stress of the exercise program in response to determining that the athletic metric improved over a duration of time of the exercise program.

8. A method comprising:
receiving physiological data captured by sensors of a garment worn by a user, the physiological data describing muscle activation of a set of muscles of the user while performing an exercise;
determining, for the set of muscles, a muscle stress measurement representative of a magnitude of exertion by the set of muscles during the exercise; and
accessing a pre-determined muscle stress measurement model, the pre-determined muscle stress measurement model defining one or more criteria for determining risk of injury;
in response to at least one criteria defined by the pre-determined muscle stress measurement model being satisfied by the muscle stress measurement, notifying the user of the risk of injury via a client device.

9. The method of claim 8, further comprising:
determining that the at least one criteria defined by the pre-determined muscle stress measurement model is satisfied by determining that muscle stress measurement exceeds a threshold deviation relative to athlete population norms.

10. The method of claim 8, further comprising:
determining the risk of injury by identifying an imbalance of muscle stress of the user in response to the at least one criteria defined by the pre-determined muscle stress measurement model being satisfied by the muscle stress measurement.

11. The method of claim 10, wherein identifying the imbalance of muscle stress comprises:
determining that muscle stress of a left body side or right body side is greater than muscle stress of the other side by at least a threshold difference.

12. A method comprising:
receiving physiological data captured by sensors of a garment worn by a user, the physiological data describing muscle activation of a set of muscles of the user while performing an exercise;
determining, for the set of muscles, a muscle stress measurement representative of a magnitude of exertion by the set of muscles during the exercise;
generating biofeedback based at least in part on the determined muscle stress measurement; and
presenting the generated biofeedback via a client device.

13. The method of claim 12, further comprising:
identifying an imbalance of muscle stress by determining that muscle stress of a left body side or right body side is greater than muscle stress of the other side by at least a threshold difference; and
wherein generating the biofeedback is further based on the imbalance of muscle stress.

14. The method of claim 12, wherein the biofeedback includes a comparison between the user and another user of a same athletic team as the user, and wherein the generated biofeedback is presented via the client device to a coach of the same athletic team.

15. The method of claim 12, wherein the biofeedback includes a comparison between a contribution of the set of muscles and another contribution of a different set of muscles of the user while performing the exercise.

16. The method of claim 12, further comprising:
receiving heart rate data captured by the sensors of the garment; and
wherein generating the biofeedback is further based on the heart rate data.

17. The method of claim 12, wherein the biofeedback indicates a measure of progress of the user over a period of time, the biofeedback generated further based on context describing the exercise.

18. A computer program product comprising a non-transitory computer readable storage medium having instructions encoded thereon that, when executed by a processor, cause the processor to:
receive physiological data captured by sensors of a garment worn by a user, the physiological data describing muscle activation of a set of muscles of the user while performing an exercise in an exercise program presented to the user by a client device;
determine, for the set of muscles, a muscle stress measurement representative of a magnitude of exertion by the set of muscles during the exercise;
access a pre-determined muscle stress measurement model, the pre-determined muscle stress measurement model defining one or more criteria for modifying the exercise program;
in response to at least one criteria defined by the pre-determined muscle stress measurement model being satisfied by the muscle stress measurement, modify the exercise program; and
present to the user, by the client device, information representative of the modified exercise program.

19. The non-transitory computer readable storage medium of claim 18, having further instructions that when executed by the processor cause the processor to:
determine that the at least one criteria defined by the pre-determined muscle stress measurement model is satisfied by determining that muscle stress measurement exceeds a threshold deviation relative to athlete population norms.

20. The non-transitory computer readable storage medium of claim 18, having further instructions that when executed by the processor cause the processor to:
determine an imbalance of muscle stress of the user in response to the at least one criteria defined by the pre-determined muscle stress measurement model being satisfied by the muscle stress measurement; and
wherein modifying the exercise program comprises adding or removing one or more exercises of the exercise program to address the imbalance of muscle stress.
